(19) United States
(12) Patent Application Publication
(10) Pub. No.: US 2007/0202101 A1
(43) Pub. Date: Aug. 30, 2007

(54) Combination therapy using anti-EGFR antibodies and anti-hormonal agents

(75) Inventors: Oliver Rosen, Darmstadt (DE); Andreas Harstrick, Gross-Umstadt (DE)

Correspondence Address: Kirkpatrick & Lockhart Preston Gates Ellis LLP;(FORMERLY KIRKPATRICK & LOCKHART NICHOLSON GRAHAM), STATE STREET FINANCIAL CENTER, One Lincoln Street, BOSTON, MA, 02111-2950, US

(73) Assignee: Merck Patent GmbH, Darmstadt (DE)

(21) Appl. No.: 11/789,883

(22) Filed: Apr. 26, 2007

Related U.S. Application Data
(62) Division of application No. 10/476,478, filed on Oct. 31, 2003.

(30) Foreign Application Priority Data
May 8, 2001 (EP) 01111049.1

(51) Int. Cl. A61K 39/395; A61K 31/56; A61K 38/09

(52) U.S. Cl. 424/143.1; 514/15; 514/171

(57) ABSTRACT

The invention relates to a combination therapy for the treatment of tumors and tumor metastases, preferably breast and prostate tumors, comprising administration of anti-EGFR (Her1) antibodies and anti-hormonal agents, optionally together with cytotoxic/chemotherapeutic agent. The method and the pharmaceutical compositions comprising said agents can result in a synergistic potentiation of the tumor cell proliferation inhibition effect of each individual therapeutic agent, yielding more effective treatment than found by administering an individual component alone.

### TECHNICAL FIELD OF THE INVENTION

The invention relates to a combination therapy for the treatment of tumors and tumor metastases, preferably breast and prostate tumors, comprising administration of anti-EGFR (Her1) antibodies and anti-hormonal agents, optionally together with cytotoxic/chemotherapeutic agents. The method and the pharmaceutical compositions comprising said agents can result in a synergistic potentiation of the tumor cell proliferation inhibition effect of each individual therapeutic agent, yielding more effective treatment than found by administering an individual component alone.

### BACKGROUND OF THE INVENTION

Tyrosine kinases are a class of enzymes that catalyze the transfer of the terminal phosphate of adenosine triphosphate to tyrosine residues in protein substrates.

Tyrosine kinases are believed, by way of substrate phosphorylation, to play critical roles in signal transduction for a number of cell functions. Though the exact mechanisms of signal transduction is still unclear, tyrosine kinases have been shown to be important contributing factors in cell proliferation, carcinogenesis and cell differentiation.

Tyrosine kinases can be categorized as receptor type or non-receptor type. Both receptor-type and non-receptor type tyrosine kinases are implicated in cellular signaling pathways leading to numerous pathogenic conditions, including cancer, psoriasis and hyperimmune responses. Many tyrosine kinases are involved in cell growth as well as in angiogenesis.

The non-receptor type of tyrosine kinases is also comprised of numerous subfamilies, including Src, Frk, Btk, Csk, Abl, Zap70, Fes/Fps, Fak, Jak, Ack, and LIMK. Each of these subfamilies is further sub-divided into varying receptors. For example, the Src subfamily is one of the largest and includes Src, Yes, Fyn, Lyn, Lck, Blk. Hck, Fgr, and Yrk. The Src subfamily of enzymes has been linked to oncogenesis. For a more detailed discussion of the nor-receptor type of tyrosine kinases, see Bolen Oncogene, 8:2025-2031 (1993).

Receptor type tyrosine kinases have an extracellular, a transmembrane, and an intracellular portion, while non-receptor type tyrosine kinases are wholly intracellular. Receptor-linked tyrosine kinases are transmembrane proteins that contain an extracellular ligand binding domain, a transmembrane sequence, and a cytoplasmic tyrosine kinase domain. The receptor-type tyrosine kinases are comprised of a large number of transmembrane receptors with diverse biological activity. In fact, different subfamilies of receptor-type tyrosine kinases have been identified. Implicated tyrosine kinases include fibroblast growth factor (FGF) receptors, epidermal growth factor (EGF) receptors of the ErbB major class family, and platelet-derived growth factor (PDGF) receptors. Also implicated are nerve growth Factor (NGF) receptors, brain-derived neurotrophic Factor (BDNF) receptors, and neurotrophin-3 (NT-3) receptors, and neurotrophin-4 (NT-4) receptors.

One receptor type tyrosine kinase subfamily, designated as HER or ErbB subfamily, is comprised of EGFR (ErbB1), HER2 (ErbB2 or p185neu), HER3 (ErbB3), and HER4(ErbB4 or tyro2). Ligands of this subfamily of receptors include epithelial growth factor (EGF), TGF-a, amphiregulin, HB-EGF, betacellulin and heregulin. The PDGF subfamily includes the FLK family which is comprised of the kinase insert domain receptor (KDR).

EGFR, encoded by the erbB1 gene, has been causally implicated in human malignancy. In particular, increased expression of EGFR has been observed in breast, bladder, lung, head, neck and stomach cancer as well as glioblastomas. Increased EGFR receptor expression is often associated with increased production of the EGFR ligand, transforming growth factor alpha (TGF-a), by the same tumor cells resulting in receptor activation by an autocrine stimulatory pathway (Baselga and Mendelsohn, Pharmac. Ther. 64:127-154 (1994)). The EGF receptor is a transmembrane glycoprotein which has a molecular weight of 170.000, and is found on many epithelial cell types. It is activated by at least three ligands, EGF, TGF-α (transforming growth factor alpha) and amphiregulin. Both epidermal growth factor (EGF) and transforming growth factor-alpha (TGF-α) have been demonstrated to bind to EGF receptor and to lead to cellular proliferation and tumor growth. These growth factors do not bind to HER2 (Ulrich and Schlesinger, 1990, Cell 61, 203). In contrary to several families of growth factors, which induce receptor dimerization by virtue of their dimeric nature (e.g. PDGF) monomeric growth factors, such as EGF, contain two binding sites for their receptors and, therefore, can cross-link two neighboring EGF receptors (Lemmon et al., 1997, EMBO J. 16, 281). Receptor dimerization is essential for stimulating of the intrinsic catalytic activity and for the auto-phosphorylation of growth factor receptors. It should be remarked that receptor protein tyrosine kinases (PTKs) are able to undergo both homo- and heterodimerization.

It has been demonstrated that anti-EGF receptor antibodies while blocking EGF and TGF-a binding to the receptor appear to inhibit tumor cell proliferation. In view of these findings, a number of murine and rat monoclonal antibodies against EGF receptor have been developed and tested for their ability inhibit the growth of tumor cells in vitro and in vivo (Modjtahedi and Dean, 1994, J. Oncology 4, 277). Humanized monoclonal antibody 425 (hMAb 425, U.S. Pat. No. 5,558,864; EP 0531 472) and chimeric monoclonal antibody 225 (cMAb 225, U.S. Pat. No. 4,943,533 and EP 0359 282), both directed to the EGF receptor, have shown their efficacy in clinical trials. The C225 antibody was demonstrated to inhibit EGF-mediated tumor cell growth in vitro and inhibit human tumor formation in vivo in nude mice. The antibody, moreover, appeared to act, above all, in synergy with certain chemotherapeutic agents (i.e., doxorubicin, adriamycin, taxol, and cisplatin) to eradicate human tumors in vivo in xenograft mouse models. Ye et al. (1999, Oncogene 18, 731) have reported that human ovarian cancer cells can be treated successfully with a combination of both cMAb 225 and humanized MAb 4D5 which is directed to the HER2 receptor.

The second member of the ErbB family, HER2 (ErbB2 or p185neu), was originally identified as the product of the transforming gene from neuroblastomas of chemically treated rats. The activated form of the neu proto-oncogene results from a point mutation (valine to glutamic acid) in the transmembrane region of the encoded protein. Amplification of the human homolog of neu is observed in breast and ovarian cancers and correlates with a poor prognosis (Slamon et al., Science, 235: 177-182 (1987); Slamon et al., Science, 244:707-712 (1989); U.S. Pat. No. 4,968,603). ErbB2 (HER2) has a molecular weight of about 185.000, with considerable homology to the EGF receptor (HER1), although a specific ligand for HER2 has not yet been clearly identified so far.

The antibody 4D5 directed to the HER2 receptor, was further found to sensitize ErbB2-overexpressing breast tumor cell lines to the cytotoxic effects of TNFα (U.S. Pat. No. 5,677,171). A recombinant humanized version of the murine anti-ErbB2 antibody 4D5 (huMAb4D5-8, rhuMAb HER2 or HERCEPTIN®; U.S. Pat. No. 5,821,337) is clinically active in patients with ErbB2-overexpressing metastatic breast cancers that have received extensive prior anti-cancer therapy (Baselga et al., J. Clin. Oncol. 14:737-744 (1996)). HERCEPTIN® received marketing approval in 1998 for the treatment of patients with metastatic breast cancer whose tumors overexpress the ErbB2 protein.

Prostate and breast cancer are the most frequently diagnosed cancers in men/women and are responsible for more than several hundred thousands deaths worldwide annually. Early stage, organ-confined, prostate and breast cancer is often managed with surgery or radiation therapy until the patient dies from unrelated causes. Carcinomas such as breast cancer, colon cancer and adenocarcinoma are characterized by rapid cell division. Consequently, these cancers are amenable to treatment with chemotherapeutic agents that inhibit rapid cell division. In contrast, prostate cancer is not characterized by rapid cell division. Therefore, conventional chemotherapeutic agents generally display low efficacy against prostatic carcinomas. Prostatic carcinomas are often sensitive to hormonal manipulation. Currently approved treatment of prostrate cancer includes surgical castration, chemical castration, or a combination of surgical and chemical castration.

Removal of the testes, the primary testosterone producing organ, reduces the levels of circulating androgens, to less than 5% of normal levels. This reduction in androgen levels inhibits prostate tumor growth. Although the anti-tumor effects of surgical castration are direct, the anti-tumor effects can be temporary. Surgical castration often leads to clonal selection of androgen-independent prostate tumor cells. This results in re-growth of the prostate tumor in a form that proliferates without testosterone or DHT Stimulation (Isaacs et al. (1981) Cancer Res. 41:5070-5075; Crawford et al. (1989) IV. Eng. J. Med. 321:419-424). Chemical castration (also called medical castration) is often substituted for surgical castration, as an initial treatment.

Prostate and breast cancer share a unique feature which is that in the majority they are stimulated in growth by steroid sex hormones (estrogenes for breast cancer and androgenes for prostate cancer, respectively). The steroid sex hormones deliver their stimulating signals by binding to specific receptors. Estrogene and androgene receptors can be found on approximately 75% of breast cancer cells and nearly 100% of prostate cancer cells and are members of the nuclear hormone receptor superfamily which includes the receptors for steroid hormones, thyroid hormones, lipophilic vitamins such as vitamins A and D, and the orphan receptors, which have a structure consistent with other superfamily members but have no identified ligands (Evans (1988) Science 240:889-895). The receptors regulate gene expression by interacting with specific DNA sequences in the promoters of target genes (Glass (1994) Endocr. Rev. 15:391-407).

Nuclear receptors are grouped into two subfamilies: the thyroidiretinoic acid/vitamin D receptor (TRV) family and the steroid receptor (R_{S}) family. Steroid hormone receptors bind to their respective HREs in a ligand-dependent manner whereas some receptors such as the thyroid hormone receptor (T3R) and retinoic acid receptor (RAR/RXR) bind to their response elements in a ligand-independent manner. Steroid receptors play a role in normal health and in a spectrum of disease states, including cancer, inflammation, endocrine disorders, and oral contraception. The natural steroid hormones produced by endocrine glands bind to steroid hormone receptors in target organs.

The natural steroid hormones include estrogens, progestins, androgens, glucocorticoids and mineralocorticoids. These hormones are defined as agonists, and hormone-receptor complexes modulate specific gene transcription by either increasing or decreasing transcription rate.

Steroid agonists have pleiotropic physiological actions in a number of tissues, for example, estradiol and progesterone regulate gene transcription in the kidney, ovary, cervix, uterus, bone, skin, breast, heart, pituitary and brain. Hormones of the steroid receptor subfamily are used to treat many disorders and are used in healthy people for oral contraception and hormone replacement therapy, among others.

It is often medically desirable to block the actions of steroid hormone agonists. For this reason, researchers have synthesized steroid receptor antagonists that are used in breast, endometrial and prostate cancer treatment as agents to prevent cancer development or block abnormal growths, and as contraceptive agents. These antagonists are ligands also to the steroid receptors, but in general, they have effects that are opposite to the ones produced by the agonists.

The actions of steroid receptor antagonist are complex. They often have dual agonist/antagonist effects. For example, an antagonist may have partially the biological activity of an agonist; thus, the antagonist may block the activity of the agonist, resulting in substantially decreased agonist activity. Antagonists may also have the desired antagonist effect in one tissue (for example, the breast), but may have an agonist effect in another tissue (for example, the Uterus). The agonist effect of an antagonist may or may not be an unwanted side-effect. Similarly, in cancer treatment, an antagonist ligand may initially have the desired inhibitory effect on the tumor, but with time, the ligand switches to an agonist-like effect and the cancer then resumes growing.

A typical example is LHRH (luteinizing hormone-releasing hormone). LHRH active compounds administered in individual single higher doses stimulate the hormone production (agonist action), whereas continuous small doses of said compounds act as antagonists inhibiting the hormone releasing effect

Anglogenesis, also referred to as neovascularization, is a process of tissue vascularization that involves the growth of new developing blood vessels into a tissue. The process is mediated by the infiltration of endothelial cells and smooth muscle cells. The process is believed to proceed in any one of three ways: (1) The vessels can sprout from pre-existing vessels; (2) De novo development of vessels can arise from precursor cells (vasculogenesis); or (3) Existing small vessels can enlarge in diameter (Blood et al., 1990, Bioch. Biophys. Acta 1032, 89. Vascuiar endothelial cells are known to contain at least five RGD-dependent integrins, including the vitronectin receptor (αᵥβ₃ or αᵥβ₅), the collagen Types I and IV receptor, the laminin receptor, the fibronectin/laminin/collagen receptor and the fibronectin receptor (Davis et al., 1993, J. Cell. Biochem. 51, 206). The smooth muscle cell is known to contain at least six RGD-dependent integrins, including αᵥβ₃ αᵥβ₅.

Angiogenesis is an important process in neonatal growth, but is also important in wound healing and in the pathogenesis of a large variety of clinically important diseases including tissue inflammation, arthritis, psoriasis, cancer, diabetic retinopathy, macular degeneration and other neovascular eye diseases. These clinical entities associated with angiogenesis are referred to as angiogenic diseases (Folkman et al., 1987, Science 235, 442).

Inhibition of cell adhesion in vitro using monoclonal antibodies immunospecific for various integrin α or β subunits have implicated the vitronectin receptor αᵥβ₃ in cell adhesion of a variety of cell types including microvascular endothelial cells (Davis et al., 1993, J. Cell. Biol. 51, 206).

Integrins are a class of cellular receptors known to bind extracellular matrix proteins, and therefore mediate cell-cell and cell-extracellular matrix interactions, referred generally to as cell adhesion events. The integrin receptors constitute a family of proteins with shared structural characteristics of noncovalent heterodimeric glycoprotein complexes formed of α and β subunits. The vitronectin receptor, named for its original characteristic of preferential binding to vitronectin, is now known to refer to three different integrins, designated αᵥβ₁, αᵥβ₃ and αᵥβ₅. αᵥβ₁ binds fibronectin and vitronectin. αᵥβ₃ binds a large variety of ligands, including fibrin, fibrinogen, laminin, thrombospondin, vitronectin and von Willebrand's factor. αᵥβ₅ binds vitronectin. It is clear that there are different integrins with different biological functions as well as different integrins and subunits having shared biological specificity. One important recognition site in a ligand for many integrins is the arginine-glycine-aspartic acid (RGD) tripeptide sequence. RGD is found in all of the ligands identified above for the vitronectin receptor integrins.

This RGD recognition site can be mimicked by linear and cyclic (poly)peptides that contain the RGD sequence. Such RGD peptides are known to be inhibitors or antagonists, respectively, of integrin function. It is important to note, however, that depending upon the sequence and structure of the RGD peptide, the specificity of the inhibition can be altered to target specific integrins. Various RGD polypeptides of varying integrin specificity have been described, for example, by Cheresh, et al., 1989, Cell 58, 945, Aumailley et al., 1991, FEBS Letts. 291, 50, and in numerous patent applications and patens (e.g. U.S. Pat. Nos. 4,517,686, 4,578,079, 4,589,881, 4,614,517, 4,661,111, 4,792,525; EP 0770 622).

The generation of new blood vessels, or angiogenesis, plays a key role in the growth of malignant disease and has generated much interest in developing agents that inhibit angiogenesis (see, for example, Holmgren et al., 1995, Nature Medicine 1, 149; Folkman, 1995, Nature Medicine 1, 27; O'Reilly et. al., 1994, Cell 79, 315). The use of αᵥβ₃ integrin antagonists to inhibit angiogenesis is known in methods to inhibit solid tumor growth by reduction of the blood supply to the solid tumor (see, for example, U.S. Pat. No. 5,753,230 and U.S. Pat. No. 5,766,591, which describe the use of αᵥβ₃ antagonists such as synthetic polypeptides, monoclonal antibodies and mimetics of αᵥβ₃ that bind to the αᵥβ₃ receptor and inhibit angiogenesis). Methods and compositions for inhibiting αᵥβ₅ mediated angiogenesis of tissues using antagonists of the vitronectin receptor αᵥβ₅ are disclosed in WO 97/45447.

Angiogenesis is characterized by invasion, migration and proliferation of endothelial cells, processes that depend on cell interactions with extracellular matrix components. In this context, the integrin cell-matrix receptors mediate cell spreading and migration. The endothelial adhesion receptors of integrin αᵥβ₃ was shown to be a key player by providing a vasculature-specific target for anti-angiogenic treatment strategies (Brooks et al., 1994, Science 264, 569; Friedlander et. al., 1995, Science 270). The requirement for vascular integrin αᵥβ₃ in angiogenesis was demonstrated by several in vivo models where the generation of new blood vessels by transplanted human tumors was entirely inhibited either by systemic administration of peptide antagonists of integrin αᵥβ₃ and αᵥβ₅, as indicated above, or, alternatively, by anti-αᵥβ₃ antibody LM609 (Brooks et al., 1994, Cell 79, 1157; ATCC HB 9537). This antibody blocks the αᵥβ₃ integrin receptor the activation of which by its natural ligands promotes apoptosis of the proliferative angiogenic vascular cells and thereby disrupts the maturation of newly forming blood vessels, an event essential for the proliferation of tumors. Nevertheless, it was recently reported, that melanoma cells could form web-like patterns of blood vessels even in the absence of endothelial cells (1999, Science 285, 14), implying that tumors might be able to circumvent some anti-angiogenic drugs which are only effective in the presence of endothelial tissue.

Numerous molecules stimulate endothelial proliferation, migration and assembly, including VEGF, Ang1 and bFGF, and are vital survival factors. VEGF (Vascular Endothelial Growth Factor) has been identified as a selective angiogenic growth factor that can stimulate endothelial cell mitogenesis. VEGF, in particular, is thought to be a major mediator of angiogenesis in a primary tumor and in ischemic ocular diseases. VEGF is a homodimer (MW: 46.000) that is an endothelial cell-specific angiogenic (Ferrara et al., 1992, Endocrin. Rev., 13, 18) and vasopermeable factor (Senger et al., 1986, Cancer Res., 465629) that binds to high-affinity membrane-bound receptors with tyrosine kinase activity (Jakeman et al., 1992, J. Clin. Invest., 89, 244). Human tumor biopsies exhibit enhanced expression of VEGF mRNAs by malignant cells and VEGF receptor mRNAs in adjacent endothelial cells. VEGF expression appears to be greatest in regions of tumors adjacent to vascular areas of necrosis. (for review see Thomas et al., 1996, J. Biol. Chem. 271(2), 603; Folkman, 1995, Nature Medicine 1, 27). WO 97/45447 has implicated the αᵥβ₅ integrin in neovascularization, particularly, that induced by VEGF, EGF and TGF-α, and discloses that αᵥβ₅ antagonist can inhibit VEGF promoted angiogenesis. Effective anti-tumor therapies may also utilize targeting VEGF receptor for inhibition of angiogenesis using monoclonal antibodies. (Witte et al.,1998, Cancer Metastasis Rev. 17(2), 155). MAb DC-101 is known to inhibit angiogenesis of tumor cells.

The present invention describes now that antibodies specifically directed to EGF receptor (ErbB1, Her1) are more effective in killing or shrinking tumor tissue of especially prostate and breast cancer when administered together with anti-hormonal agents, especially inhibitors of the nuclear hormone receptor family. Further co-administration with anti-angiogenic agents and/or cytotoxic agents may improve the positive and synergistic effect of said combination therapy.

### SUMMARY OF THE INVENTION

The present inventions describes for the first time the new concept in tumor therapy to administer to an individual an agent that blocks or inhibits the EGF receptor together with an anti-hormonal agent. Optionally the composition according to this invention comprises further therapeutically active compounds, preferably selected from the group consisting of cytotoxic agents, chemotherapeutic agents and inhibitors or antagonists of the ErbB receptor tyrosine kinase family or inhibitors or antagonist of angiogenesis.

Thus, the invention relates to pharmaceutical compositions comprising as preferred anti-EGFR agent an anti-EGFR antibody and as anti-hormonal agent an inhibitor or antagonist of the nuclear receptor, preferably an steroid receptor. According to this invention said therapeutically active agents may also be provided by means of a pharmaceutical kit comprising a package comprising one or more anti-EGFR antibody, one or more anti-hormonal agents, and, optionally, one or more cytotoxic/chemotherapeutic agents, anti-ErbB agents, anti-angiogenic agents in single packages or in separate containers. The therapy with this combinations may include optionally treatment with radiation.

However, the invention relates, furthermore, to a combination therapy comprising the administration of only one (fusion) molecule, having anti-EGFR activity and anti-hormonal activity, optionally together with one or more cytotoxic/chemotherapeutic agents. An example is an anti-EGFR antibody, such as h425 or c225 as described above and below, which is fused at the C-terminal of its Fc portion to an anti-hormonal agent by known recombinant or chemical methods.

A further example is a bispecific antibody, wherein one specificity is directed to an nuclear hormone receptor and the other one is directed to the EGF receptor.

Principally, the administration can be accompanied by radiation therapy, wherein radiation treatment can be done substantially concurrently or before or after the drug administration. The administration of the different agents of the combination therapy according to the invention can also be achieved substantially concurrently or sequentially. Tumors, bearing receptors on their cell surfaces involved in the development of the blood vessels of the tumor, may be successfully treated by the combination therapy of this invention.

It is known that tumors elicit alternative routes for their development and growth. If one route is blocked they often have the capability to switch to another route by expressing and using other receptors and signaling pathways. Therefore, the pharmaceutical combinations of the present invention may block several of such possible development strategies of the tumor and provide consequently various benefits. The combinations according to the present invention are useful in treating and preventing tumors, tumor-like and neoplasia disorders and tumor metastases, which develop and grow by activation of their relevant hormone receptors which are present on the surface of the tumor cells. Preferably, the different combined agents of the present invention are administered in combination at a low dose, that is, at a dose lower than has been conventionally used in clinical situations. A benefit of lowering the dose of the compounds, compositions, agents and therapies of the present invention administered to an individual includes a decrease in the incidence of adverse effects associated with higher dosages. For example, by the lowering the dosage of an agent described above and below, a reduction in the frequency and the severity of nausea and vomiting will result when compared to that observed at higher dosages. By lowering the incidence of adverse effects, an improvement in the quality of life of a cancer patient is contemplated. Further benefits of lowering the incidence of adverse effects include an improvement in patient compliance, a reduction in the number of hospitalizations needed for the treatment of adverse effects, and a reduction in the administration of analgesic agents needed to treat pain associated with the adverse effects. Alternatively, the methods and combination of the present invention can also maximize the therapeutic effect at higher doses.

The combinations according to the inventions show an astonishing synergetic effect. In administering the combination of drugs real tumor shrinking and disintegration could be observed during clinical studies while no significant adverse drug reactions were detectable.

In detail the invention refers to:
a pharmaceutical composition comprising in an therapeutically effective amount at least (i) one anti-EGFR antibody or an immunotherapeutically effective fragment thereof and (ii) one anti-hormonal agent, optionally together with a pharmaceutically acceptable carrier, excipient or diluent;
a corresponding pharmaceutical composition, wherein said anti-EGFR antibody or said immunotherapeutically effective fragment thereof is murine, chimeric or humanized Mab 425 (h425) or chimeric Mab 225 (c225) inhibitor/antagonist;
a corresponding pharmaceutical composition, wherein said anti-hormonal agent is an inhibitor of the nuclear hormone receptor family;
a corresponding pharmaceutical composition, wherein said anti-hormonal agent is an steroid receptor inhibitor/antagonist;
a corresponding pharmaceutical composition comprising additionally an anti-angiogenic agent;
a corresponding pharmaceutical composition further comprising a cytotoxic and/or chemotherapeutic agent;
a corresponding pharmaceutical composition comprising additionally a further an anti-HER2 antibody or an immunotherapeutically active fragment thereof;
a pharmaceutical composition comprising an antibody having an anti-EGFR activity and an anti-nuclear hormone receptor activity, optionally together with a pharmaceutically acceptable carrier, excipient or diluent;
a corresponding pharmaceutical, wherein said antibody is a bispecifc antibody;
a pharmaceutical kit comprising a package comprising at least (i) one anti-EGFR antibody or an immunotherapeutically effective fragment thereof, and (ii) one anti-hormonal agent, and optionally (iii) a cytotoxic and/or chemotherapeutic agent;
a corresponding pharmaceutical kit comprising (i) monoclonal antibody h425, and (ii) a steroid receptor antagonist;
a corresponding pharmaceutical kit comprising (i) monoclonal antibody h425, and (ii) a LHRH antagonist;
a corresponding pharmaceutical kit, wherein said pharmaceutically active agents are provided in separate containers in said package;
the use of a pharmaceutical composition or a pharmaceutical kit as defined above and below for the manufacture of a medicament or a composition of medicaments to treat tumors and tumor metastases preferably for the treatment of breast and prostate cancer;
the corresponding use for the treatment of steroid-independent breast and prostate cancer;
method for treating tumors or tumor metastases in an individual comprising administering to said individual simultaneously or sequentially a therapeutically effective amount of (i) an anti-EGFR antibody, and (ii) an anti-hormonal agent;
a corresponding method, wherein said anti-EGFR antibody is monoclonal antibody h425 or c225 and said anti-hormonal agent is an steroid receptor antagonist; and
a corresponding method comprising administering additionally to said individual a therapeutically effective amount of a cytotoxic and/or chemotherapeutic agent or an anti-angiogenic agent or another anti-ErbB receptor antibody.

### DETAILED DESCRIPTION OF THE INVENTION

If not otherwise pointed out the terms and phrases used in this invention have the meanings and definitions as given below. Moreover, these definitions and meanings describe the invention in more detail, preferred embodiments included.

“Biological molecules” include natural or synthetic molecules having, as a rule, a molecular weight greater than approximately 300, and are preferably poly- and oligosaccharides, oligo- and polypeptides, proteins, peptides, poly- and oligonucleotides as well as their glycosylated lipid derivatives. Most typically, biological molecules include immunotherapeutic agents, above all antibodies or fragments thereof, or functional derivatives of these antibodies or fragments including fusion proteins.

A “receptor” or “receptor molecule” is a soluble or membrane bound/associated protein or glycoprotein comprising one or more domains to which a ligand binds to form a receptor-ligand complex. By binding the ligand, which may be an agonist or an antagonist the receptor is activated or inactivated and may initiate or block pathway signaling.

By “ligand” or “receptor ligand” is meant a natural or synthetic compound which binds a receptor molecule to form a receptor-ligand complex. The term ligand includes agonists, antagonists, and compounds with partial agonist/antagonist action.

An “agonist” or “receptor agonist” is a natural or synthetic compound which binds the receptor to form a receptor-agonist complex by activating said receptor and receptor-agonist complex, respectively, initiating a pathway signaling and further biological processes.

By “antagonist” or “receptor antagonist” is meant a natural or synthetic compound that has a biological effect opposite to that of an agonist. An antagonist binds the receptor and blocks the action of a receptor agonist by competing with the agonist for receptor. An antagonist is defined by its ability to block the actions of an agonist. A receptor antagonist may be also an antibody or an immunotherapeutically effective fragment thereof. Preferred antagonists according to the present invention are cited and discussed below.

The term “therapeutically effective” or “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The term “immunotherapeutically effective” refers to biological molecules which cause an immune response in a mammal. More specifically, the term refers to molecules which may recognize and bind an antigen. Typically, antibodies, antibody fragments and antibody fusion proteins comprising their antigen binding sites (complementary determining regions, CDRs) are immunotherapeutically effective.

An “anti-angiogenic agent” refers to a natural or synthetic compound which blocks, or interferes with to some degree, the development of blood vessels. The anti-angiogenic molecule may, for instance, be a biological molecule that binds to and blocks an angiogenic growth factor or growth factor receptor. The preferred anti-angiogenic molecule herein binds to an receptor, preferably to an integrin receptor or to VEGF receptor. The term includes according to the invention also a prodrug of said angiogenic agent.

There are a lot of molecules having different structure and origin which elicit anti-angiogenic properties. Most relevant classes of angiogenesis inhibiting or blocking agents which are suitable in this invention, are, for example:

(i) anti-mitotics such as flurouracil, mytomycin-C, taxol;

(ii) estrogen metabolites such as 2-methoxyestradiol;

(iii) matrix metalloproteinase (MMP) inhibitors which inhibit zinc metalloproteinases (metalloproteases) (e.g. betimastat, BB16, TIMPs, minocycline, GM6001, or those described in “Inhibition of Matrix Metalloproteinases: Therapeutic Applications” (Golub, Annals of the New York Academy of Science, Vol. 878a; Greenwald, Zucker (Eds.), 1999);

(iv) anti-angiogenic multi-functional agents and factors such as IFNα (U.S. Pat. No. 4,530,901; U.S. Pat. Nos. 4,503,035; 5,231,176); angiostatin and plasminogen fragments (e.g. kringle 1-4, kringle 5, kringle 1-3 (O'Reilly, M. S. et al., Cell (Cambridge, Mass.) 79(2): 315-328, 1994; Cao et al., J. Biol. Chem. 271: 29461-29467,1996; Cao et al., J. Biol Chem 272:22924-22928, 1997); endostatin (O'Reilly, M. S. et al., Cell 88(2), 277,1997 and WO 97/15666), thrombospondin (TSP-1; Frazier,1991, Curr Opin Cell Biol 3(5): 792); platelet factor 4 (PF4);

(v) plasminogen activator/urokinase inhibitors:

(vi) urokinase receptor antagonists:

(vii) heparinases;

(viii) fumagillin analogs such as TNP-470;

(ix) tyrosine kinase inhibitors such as SUI 01 (many of the above and below—mentioned ErbB receptor antagonists (EGFR/HER2 antagonists) are also tyrosine kinase inhibitors, and may show, therefore anti-EGF receptor blocking activity which results in inhibiting tumor growth, as well as anti-angiogenic activity which results in inhibiting the development of blood vessels and endothelial cells, respectively);

(x) suramin and suramin analogs;

(xi) angiostatic steroids;

(xii) VEGF and bFGF antagonists:

(xiii) VEGF receptor antagonists such as anti-VEGF receptor antibodies (D-101);

(xiv) flk-1 and fit-1 antagonists:

(xv) cyclooxxygenase-II inhibitors such as COX-II;

(xvi) integrin antagonists and integrin receptor antagonists such as αv antagonists and αv receptor antagonists, for example, anti-αv receptor antibodies and RGD peptides. Integrin (receptor) antagonists are preferred according to this invention.

The term “integrin antagonists/inhibitors” or “integrin receptor antagonists/inhibitors” refers to a natural or synthetic molecule that blocks and inhibit an integrin receptor. In some cases, the term includes antagonists directed to the ligands of said integrin receptors (such as for αᵥβ₃: vitronectin, fibrin, fibrinogen, von Willebrand's factor, thrombospondin, laminin; for αᵥβ₅: vitronectin; for αᵥβ₁: fibronectin and vitronectin; for αᵥβ₆: fibronectin).

Antagonists directed to the integrin receptors are preferred according to the invention. Integrin (receptor) antagonists may be natural or synthetic peptides, non-peptides, peptidomimetica, immunoglobulins, such as antibodies or functional fragments thereof, or immunoconjugates (fusion proteins).

Preferred integrin inhibitors of the invention are directed to receptor of αᵥ integrins (e.g. αᵥβ₃, αᵥβ₅, αᵥβ₆ and sub-classes). Preferred integrin inhibitors are αᵥ antagonists, and in particular αᵥβ₃ antagonists. Preferred αᵥ antagonists according to the invention are RGD peptides, peptidomimetic (non-peptide) antagonists and anti-integrin receptor antibodies such as antibodies blocking αᵥ receptors.

Exemplary, non-immunological αᵥβ₃ antagonists are described in the teachings of U.S. Pat. No. 5,753,230 and U.S. Pat. No. 5,766,591. Preferred antagonists are linear and cyclic RGD-containing peptides. Cyclic peptides are, as a rule, more stable and elicit an enhanced serum half-life. The most preferred integrin antagonist of the invention is, however, cyclo-(Arg-Gly-Asp-DPhe-NMeVal) (EMD 121974, Cilengitide®, Merck KgaA, Germany; EP 0770 622) which is efficacious in blocking the integrin receptors αᵥβ₃, αᵥβ₁, αᵥβ₆, αᵥβ₈, α_{IIb}β₃.

Suitable peptidic as well as peptido-mimetic (non-peptide) antagonists of the αᵥβ₃/αᵥβ₅/αᵥβ₆ integrin receptor have been described both in the scientific and patent literature. For example, reference is made to Hoekstra and Poulter, 1998, Curr. Med. Chem. 5,195; WO 95/32710; WO 95/37655; WO 97/01540; WO 97/37655; WO 97/45137; WO 97/41844; WO 98/08840; WO 98118460; WO 98/18461; WO 98/25892; WO 98/31359; WO 98/30542; WO 99/15506; WO 99/15507; WO 99/31061; WO 00/06169; EP 0853 084; EP 0854 140; EP 0854 145; U.S. Pat. No. 5,780,426; and U.S. Pat. No. 6,048,861. Patents that disclose benzazepine, as well as related benzodiazepine and benzocycloheptene αᵥβ₃ integrin receptor antagonists, which are also suitable for the use in this invention, include WO 96/00574, WO 96/00730, WO 96/06087, WO 96/26190, WO 97/24119, WO 97/24122, WO 97/24124, WO 98/15278, WO 99/05107, WO 99/06049, WO 99/15170, WO 99/15178, WO 97/34865, WO 97/01540, WO 98/30542, WO 99/11626, and WO 99/15508. Other integrin receptor antagonists featuring backbone conformational ring constraints have been described in WO 98/08840; WO 99/30709; WO 99/30713; WO 99/31099; WO 00/09503; U.S. Pat. No. 5,919,792; U.S. Pat. No. 5,925,655; U.S. Pat. No. 5,981,546; and U.S. Pat. No. 6,017,926. In U.S. Pat. No. 6,048,861 and WO 00/72801 a series of nonanoic acid derivatives which are potent αᵥβ₃ integrin receptor antagonists were disclosed. Other chemical small molecule integrin antagonists (mostly vitronectin antagonists) are described in WO 00/38665. Other αᵥβ₃ receptor antagonists have been shown to be effective in inhibiting angiogenesis. For example, synthetic receptor antagonists such as (S)-10,11-Dihydro-3-[3-(pyridin-2-ylamino)-1-propyloxy]-5H-dibenzo[a,d]cycloheptene-10-acetic acid (known as SB-265123) have been tested in a variety of mammalian model systems. (Keenan et al., 1998, Bioorg. Med. Chem. Lett. 8(22), 3171; Ward et al., 1999, Drug Metab. Dispos. 27(11),1232). Assays for the identification of integrin antagonists suitable for use as an antagonist are described, e.g. by Smith et al., 1990, J. Biol. Chem. 265, 12267, and in the referenced patent literature.

Anti-integrin receptor antibodies are also well known. Suitable anti-integrin (e.g. αᵥβ₃, αᵥβ₅, αᵥβ₆) monoclonal antibodies can be modified to encompasses antigen binding fragments thereof, including F(ab)₂, Fab, and engineered Fv or single-chain antibody. One suitable and preferably used monoclonal antibody directed against integrin receptor αᵥβ₃ is identified as LM609 (Brooks et al., 1994, Cell 79, 1157; ATCC HB 9537). A potent specific anti-αᵥβ₅ antibody, P1F6, is disclosed in WO 97/45447, which is also preferred according to this invention. A further suitable αᵥβ₆ selective antibody is MAb 14D9.F8 (WO 99/37683, DSM ACC2331, Merck KGBA, Germany) as well as MAb 17.E6 (EP 0719 859, DSM ACC2160, Merck KGBA) which is selectively directed to the αᵥ-chain of integrin receptors. Another suitable anti-integrin antibody is the commercialized Vitraxin®.

A “angiogenic growth factor or growth factor receptor” is a factor or receptor which promotes by its activation the growth and development of blood vessels. Typically, Vascular Endothelial Growth Factor (VEGF) and its receptor belong to this group.

The term “antibody” or “immunoglobulin” herein is used in the broadest sense and specifically covers intact monoclonal antibodies, polyclonal antibodies, multispecific antibodies (e.g. bispecific antibodies) formed from at least two intact antibodies, and antibody fragments, so long as they exhibit the desired biological activity. The term generally includes heteroantibodies which are composed of two or more antibodies or fragments thereof of different binding specificity which are linked together.

Depending on the amino acid sequence of their constant regions, intact antibodies can be assigned to different “antibody (immunoglobulin) classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ and μ respectively. Preferred major class for antibodies according to the invention is IgG, in more detail IgG1 and IgG2.

Antibodies are usually glycoproteins having a molecular weight of about 150,000, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. Methods for making monoclonal antibodies include the hybridoma method described by Kohler and Milstein (1975, Nature 256, 495) and in “Monoclonal Antibody Technology, The Production and Characterization of Rodent and Human Hybridomas” (1985, Burdon et al., Eds, Laboratory Techniques in Biochemistry and Molecular Biology, Volume 13, Elsevier Science Publishers, Amsterdam), or may be made by well known recombinant DNA methods (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:58, 1-597(1991), for example.

The term “chimeric antibody” means antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g.: U.S. Pat. No. 4,816,567; Morrison et al., Proc. Nat. Acad. Sci. USA, 81:6851-6855 (1984)). Methods for making chimeric and humanized antibodies are also known in the art. For example, methods for making chimeric antibodies include those described in patents by Boss (Celitech) and by Cabilly (Genentech) (U.S. Pat. No. 4,816,397; U.S. Pat. No. 4,816,567).

“Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

The term “Variable” or “FR” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs (FR1-FR4), largely adopting a β-sheet configuration, connected by three hypervariable regions, which form loops connecting, and in some cases forming part of the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” or “CDR” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)).

“Framework Region” or “FR” residues are those variable domain residues other than the hypervariable region residues as herein defined.

“Antibody fragments” comprise a portion of an intact antibody, preferably comprising the antigen-binding or variable region thereof. Examples of antibody fragments include Fab, Fab′, F(ab′)2, Fv and Fc fragments, diabodies, linear antibodies, single-chain antibody molecules; and multispecific antibodies formed from antibody fragment(s). An “intact” antibody is one which comprises an antigen-binding variable region as well as a light chain constant domain (CL) and heavy chain constant domains, CH1, CH2 and CH3. Preferably, the intact antibody has one or more effector functions.

Papain digestion of antibodies produces two Identical antigen-binding fragments, called “Fab” fragments, each comprising a single antigen-binding site and a CL and a CH1 region, and a residual “Fc” fragment, whose name reflects its ability to crystallize readily.

The “Fc” region of the antibodies comprises, as a rule, a CH2, CH3 and the hinge region of an IgG1 or IgG2 antibody major class. The hinge region is a group of about 15 amino acid residues which combine the CH1 region with the CH2-CH3 region.

Pepsin treatment yields an “F(ab′)2” fragment that has two antigen-binding sites and is still capable of cross-linking antigen. “Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions (CDRs) of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. The Fab fragment also contains the constant domain of the light chain and the first constant domain (CH1) of the heavy chain. “Fab′” fragments differ from Fab fragments by the addition of a few residues at the carboxy terminus of the heavy chain CH1 domain including one or more cysteines from the antibody hinge region. F(ab′)2 antibody fragments originally were produced as pairs of Fab′ fragments which have hinge cysteines between them. Other chemical couplings of antibody fragments are also known (see e.g. Hermanson, Bioconjugate Techniques, Academic Press, 1996; U.S. Pat. No. 4,342,566).

“Single-chain Fv” or “scFv” antibody fragments comprise the V, and V, domains of antibody, wherein these domains are present in a Single polypeptide chain. Preferably, the Fv polypeptide further comprises a polypeptide linker between the VH and VL domains which enables the scFv to form the desired structure for antigen binding. Single-chain FV antibodies are known, for example, from Plückthun (The Pharmacology of Monoclonal Antibodies, Vol. 113, Rosenburg and Moore eds., Springer-Verlag, New York, pp. 269-315 (1994)), WO93/16185; U.S. Pat. No. 5,571,894; U.S. Pat. No. 5,587,458; Huston et al. (1988, Proc. Natl. Acad. Sci. 85, 5879) or Skerra and Plueckthun (1988, Science 240, 1038).

The term “diabodies” refers to small antibody fragments with two antigen-binding sites, which fragments comprise a variable heavy domain (V,) connected to a variable light domain (V,) in the same polypeptide chain (V,-V,). By using a linker that is too short to allow pairing between the two domains on the same chain, the domains are forced to pair with the complementary domains of another chain and create two antigen-binding sites. Diabodies are described more fully in, for example, EP 404,097; WO 93/11161.

“Bispecific antibodies” are single, divalent antibodies (or immunotherapeutically effective fragments thereof) which have two differently specific antigen binding sites. For example the first antigen binding site is directed to an angiogenesis receptor (e.g. integrin or VEGF receptor), whereas the second antigen binding site is directed to an ErbB receptor (e.g. EGFR or HER2). Bispecific antibodies can be produced by chemical techniques (see e.g., Kranz et al. (1981) Proc. Natl. Acad. Sci. USA 78, 5807), by “polydoma” techniques (See U.S. Pat. No. 4,474,893) or by recombinant DNA techniques, which all are known per se. Further methods are described in WO 91/00360, WO 92/05793 and WO 96/04305. Bispecific antibodies can also be prepared from single chain antibodies (see e.g., Huston et al. (1988) Proc. Natl. Acad. Sci. 85, 5879; Skerra and Plueckthun (1988) Science 240, 1038). These are analogues of antibody variable regions produced as a single polypeptide chain. To form the bispecific binding agent, the single chain antibodies may be coupled together chemically or by genetic engineering methods known in the art. It is also possible to produce bispecific antibodies according to this invention by using leucine zipper sequences. The sequences employed are derived from the leucine zipper regions of the transcription factors Fos and Jun (Landschulz et al., 1988, Science 240, 1759; for review, see Maniatis and Abel, 1989, Nature 341, 24). Leucine zippers are specific amino acid sequences about 20-40 residues long with leucine typically occurring at every seventh residue. Such zipper sequences form amphipathic α-helices, with the leucine residues lined up on the hydrophobic side for dimer formation. Peptides corresponding to the leucine zippers of the Fos and Jun proteins form heterodimers preferentially (O'Shea et al., 1989, Science 245, 646). Zipper containing bispecific antibodies and methods for making them are also disclosed in WO 92/10209 and WO 93/11162. A bispecific antibody according the invention may be an antibody, directed to VEGF receptor and αVβ3 receptor as discussed above with respect to the antibodies having single specificity.

The term “immunoconjugate” refers to an antibody or immunoglobulin, respectively, or a immunologically effective fragment thereof, which is fused by covalent linkage to a non-immunologically effective molecule. Preferably this fusion partner is a peptide or a protein, which may be glycosylated. Said non-antibody molecule can be linked to the C-terminal of the constant heavy chains of the antibody or to the N-terminals of the variable light and/or heavy chains. The fusion partners can be linked via a linker molecule, which is, as a rule, a 3-15 amino acid residues containing peptide. Immunoconjugates according to the invention comprise preferably fusion proteins consisting of an immunoglobulin or immunotherapeutically effective fragment thereof, directed to an angiogenic receptor, preferably an integrin or VEGF receptor and TNFα or a fusion protein consisting essentially of TNFα and IFNγ or another suitable cytokine, which is linked with its N-terminal to the C-terminal of said immunoglobulin, preferably the Fc portion thereof.

The term “fusion protein” refers to a natural or synthetic molecule consisting of one ore more non-immunotherapeutically effective (non-antibody) proteins or peptides having different specificity which are fused together optionally by a linker molecule. Fusion protein according to the invention may be molecules consisting of, for example, cyclo-(Arg-Gly-Asp-DPhe-NMeVal) fused to TNFα and/or IFNγ.

“Heteroantibodies” are two or more antibodies or antibody-binding fragments which are linked together, each of them having a different binding specificity. Heteroantibodies can be prepared by conjugating together two or more antibodies or antibody fragments. Preferred heteroantibodies are comprised of cross-linked Fab/Fabα fragments. A variety of coupling or crosslinking agents can be used to conjugate the antibodies. Examples are protein A, carboiimide, N-succinimidyl-S-acetyl-thioacetate (SATA) and N-succinimidyl-3-(2-pyridyldithio) propionate (SPDP) (see e.g., Karpovsky et al. (1984) J. EXP. Med. 160, 1686; Liu et a. (1985) Proc. Natl. Acad. Sci. USA 82, 8648). Other methods include those described by Paulus, Behring Inst. Mitt., No.78,118 (1985); Brennan et a. (1985) Science 30 m:81 or Glennie et al. (1987) J. Immunol. 139, 2367. Another method uses o-phenylenedimaleimide (oPDM) for coupling three Fab′ fragments (WO 91/03493). Multispecific antibodies are in context of this invention also suitable and can be prepared, for example according to the teaching of WO 94/13804 and WO 98/50431.

Antibody “effector functions” refer to those biological activities attributable to the Fc region (a native sequence Fc region or amino acid sequence variant Fc region) of an antibody. Examples of antibody effector functions include complement dependent cytotoxicity, Fc receptor binding, antibody-dependent cell-mediated cytotoxicity (ADCC), phagocytosis; down regulation of cell surface receptors (e.,g. B cell receptor), etc.

The term “ADCC” (antibody-dependent cell-mediated cytotoxicity) refers to a cell-mediated reaction in which nonspecific cytotoxic cells that express Fc receptors (FcR) (e.g. natural killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. To assess ADCC activity of a molecule of interest, an in vitro ADCC assay, such as that described in the prior art (U.S. Pat. No. 5,500,362; U.S. Pat. No. 5,821,337) may be performed. Useful effector cells for such assays include peripheral blood mononuclear cells (PBMC) and natural killer (NK) cells.

“Human effector cells” are leukocytes which express one or more FcRs and perform effector functions. Preferably, the cells express at least FcγRIII and perform ADCC effector function. Examples of human leukocytes which mediate DCC include peripheral blood mononuclear cells (PBMC), natural killer (NK) cells, monocytes, cytotoxic T cells and neutrophils.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcRs are reviewed, for example, in Ravetch and Kinet, Annu. Rev. Immunol 9:457-92 (1991).

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; mouse gonadotropin-associated peptide; inhibin; activin; vascular endothelial growth factor (VEGF); integrin; thrombopoietin (TPO); nerve growth factors such as NGFβ; platelet-growth factor; transforming growth factors (TGFs) such as TGFα and TGFβ; erythropoietin (EPO); interferons such as IFNα, IFNβ, and IFNγ; colony stimulating factors such as M-CSF, GM-CSF and G-CSF; interleukins such as IL-1, IL-1a, IL-2, IL-3, IL-4, IL-5, L-6, IL-7, IL-8, IL-9, IL-10, IL-11, IL-12; and TNFα or TNFβ. Preferred cytokines according to the invention are interferons and TNFa.

The term “cytotoxic agent” as used herein refers to a substance that inhibits or prevents the function of cells and/or causes destruction of cells. The term is Intended to include radioactive isotopes, chemotherapeutic agents, and toxins such as enzymatically active toxins of bacterial, fungal, plant or animal origin, or fragments thereof. The term may include also members of the cytokine family, preferably IFNγ as well as anti-neoplastic agents having also cytotoxic activity.

The term “chemotherapeutic agent” or “anti-neoplastic agent” includes according to this invention chemical agents that exert anti-neoplastic effects, i.e., prevent the development, maturation, or spread of neoplastic cells, directly on the tumor cell, e.g., by cytostatic or cytotoxic effects, and not indirectly through mechanisms such as biological response modification. Suitable chemotherapeutic agents according to the invention are preferably natural or synthetic chemical compounds, but biological molecules, such as proteins, polypeptides etc. are not expressively excluded. There are large numbers of anti-neoplastic agents available in commercial use, in clinical evaluation and in pre-clinical development, which could be included in the present invention for treatment of tumors/neoplasia by combination therapy with TNFα and the anti-angiogenic agents as cited above, optionally with other agents such as EGF receptor antagonists. It should be pointed out that the chemotherapeutic agents can be administered optionally together with above-said drug combination. Examples of chemotherapeutic or agents include alkylating agents, for example, nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic antibiotics and camptothecin derivatives. Preferred chemotherapeutic agents or chemotherapy include amifostine (ethyol), cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), doxonubicin lipo (doxil), gemcitabine (gemzar), daunorubicin, daunomubicin lipo (daunoxome), procarbazine, mitomycin, cytarabine, etoposide, methotrexate, 5-fluorouracil (5-FU), vinblastne, vincristine, bleomycin, paclitaxel (taxol), docetaxel (taxotere), aldesleukin, asparaginase, busulfan, carboplatn, cladribine, carnptothecin, CPT-11,10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatinr, pipobroman, plicamycin, streptozocin, tamoxifen, teniposide, testolactone, thioguanine, thiotepa, uracil mustard, vinorelbine, chlorambucil and combinations thereof.

Most preferred chemotherapeutic agents according to the invention are cisplatin, gemcitabine, doxorubicin, paclitaxel (taxol) and bleomycin.

The terms “cancer” and “tumor” refer to or describe the physiological condition in mammals that is typically characterized by unregulated cell growth. By means of the pharmaceutical compositions according of the present invention tumors can be treated such as tumors of the breast, heart, lung, small intestine, colon, spleen, kidney, bladder, head and neck, ovary, prostate, brain, pancreas, skin, bone, bone marrow, blood, thymus, uterus, testicles, cervix, and liver. More specifically the tumor is selected from the group consisting of adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma and teratoma. In detail, the tumor is selected from the group consisting of acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangio-carcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepnthelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adeno-carcinoma, pineal cell, pituitary tumors, plasmacytoma, pseudo-sarcoma, pulmonary blastoma, renal cell carcinoma, retinoblastoma, rhabdomyo-sarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

An “ErbB receptor” is a receptor protein tyrosine kinase which belongs to the ErbB receptor family and includes EGFR(ErbB1), ErbB2, ErbB3 and ErbB4 receptors and other members of this family to be identified in the future. The ErbB receptor will generally comprise an extracellular domain, which may bind an ErbB ligand; a lipophilic transmembrane domain; a conserved intracellular tyrosine kinase domain; and a carboxyl-terminal signaling domain harboring several tyrosine residues which can be phosphorylated. The ErbB receptor may be a “native sequence” ErbB receptor or an “amino acid sequence variant” thereof. Preferably the ErbB receptor is native sequence human ErbB receptor. ErbB1 refers to the gene encoding the EGFR protein product. Mostly preferred is the EGF receptor (HER1). The expressions “ErbB1” and “HER1” are used interchangeably herein and refer to human HER1 protein. The expressions “ErbB2” and “HER2” are used interchangeably herein and refer to human HER2 protein. ErbB1 receptors (EGFR) are preferred according to this invention

“ErbB ligand” is a polypeptide which binds to and/or activates an ErbB receptor. ErbB ligands which bind EGFR include EGF, TGF-a, amphiregulin, betacellulin, HB-EGF and epiregulin.

The term “ErbB receptor antagonist/Inhibitor” refers to a natural or synthetic molecule which binds and blocks or inhibits the ErbB receptor. Thus, by blocking the receptor the antagonist prevents binding of the ErbB ligand (agonist) and activation of the agonist/ligand receptor complex. ErbB antagonists may be directed to HER1 (EGFR) or HER2. Preferred antagonists of the invention are directed to the EGF receptor (EGFR, HER1). The ErbB receptor antagonist may be an antibody or an immunotherapeutically effective fragment thereof or non-immunobiological molecules, such as a peptide, polypeptide protein. Chemical molecules are also included, however, anti-EGFR antibodies and anti-HER2 antibodies are the preferred antagonists according to the invention. Preferred antibodies of the invention are anti-Her1 and anti-Her2 antibodies, more preferably anti-Her1 antibodies. Preferred anti-Her1 antibodies are MAb 425, preferably humanized MAb 425 (hMAb 425, U.S. Pat. No. 5,558,864; EP 0531 472) and chimeric MAb 225 (cMAb 225, U.S. Pat. No. 4,943,533 and EP 0359 282). Most preferred is monoclonal antibody h425, which has shown in mono-drug therapy high efficacy combined with reduced adverse and side effects. Most preferred anti-HER2 antibody is HERCEPTIN® commercialized by Genentech/Roche. Efficacious EGF receptor antagonists according to the invention may be also natural or synthetic chemical compounds. Some examples of preferred molecules of this category include organic compounds, organometallic compounds, salts of organic and organometallic compounds.

Examples for HER2 receptor antagonists are: styryl substituted heteroaryl compounds (U.S. Pat. No. 5,656,655); bis mono and/or bicyclic aryl heteroaryl, carbocyclic, and heterocarbocyclic compounds (U.S. Pat. No. 5,646,153); tricyclic pyrimidine compounds (U.S. Pat. No. 5,679,683); quinazoline derivatives having receptor tyrosine kinase inhibitory activity (U.S. Pat. No. 5,616,582); heteroarylethenediyl or heteroaryl-ethenediylaryl compounds (U.S. Pat. No. 5,196,446); a compound designated as 6-(2,6-dichlorophenyl)-2-(4-(2-diethyl-aminoethoxy) phenylamino)-8-methyl-8H-pyrido(2,3)-5-pyrimidin-7-one (Panek, et al., 1997, J. Pharmacol. Exp. Therap. 283, 1433) inhibiting EGFR, PDGFR, and FGFR families of receptors.

The term “tyrosine kinase antagonist/inhibitor” refers to natural or synthetic agents that are enabled to inhibit or block tyrosine kinases, receptor tyrosine kinases included. With exception of the anti-ErbB receptor antibodies mentioned above and below, more preferable tyrosine kinase antagonist agents are chemical compounds which have shown efficacy in mono-drug therapy for breast and prostate cancer. Suitable indolocarbazole-type tyrosine kinase inhibitors can be obtained using information found in documents such as U.S. Pat. Nos. 5,516,771; 5,654,427; 5,461,146; 5,650,407. U.S. Pat. Nos. 5,475,110; 5,591,855; 5,594,009 and WO 96/11933 disclose pyrrolocarbazole-type tyrosine kinase inhibitors and prostate cancer. Preferably, the dosage of the chemical tyrosine kinase inhibitors as defined above is from 1 pg/kg to 1 g/kg of body weight per day. More preferably, the dosage of tyrosine kinase inhibitors is from 0.01 mg/kg to 100 mg/kg of body weight per day.

As used herein, the term “anti-hormonal agent” includes natural or synthetic. organic or peptidic compounds that act to regulate or inhibit hormone action on tumors. In more detail an “anti-hormonal agent” (1) inhibits the production of serum androgens, (2) blocks binding of serum androgens to androgen receptors, or (3) inhibits the conversion of testosterone to DHT, or a combination of two or more such compounds.

An anti-hormonal agent according to the invention includes in general steroid receptor antagonists and in more detail anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above. The term includes also agonists and/or antagonists of glycoprotein hormones such as follicle stimulating hormone (FSH), thyroid stimulating hormone (TSH), and luteinizing hormone (LH) and LHRH (leuteinizing hormone-releasing hormone). A LHRH agonist useful in this invention is goserelin acetate, commercially available as ZOLADEX© (Zeneca). The chemical structure of goserelin acetate is pyro-Glu-His-Trp-Ser-Tyr-D-Ser(But)-Leu-Arg-Pro-Azgly-NH, acetate. An example of an LHRH antagonist useful in this invention is ANTIDE© (Ares-Serono), whose chemical name is D-alaninamide N-acetyl-3(2-naphthalenyt)-D-alanyl4 chloro-phenylalanyl-3-(3-pyridinyl)-D-alanyl-L-seryl-NG-(3-pyridinylcarbonyl)-L-lysyl-N6-(3-pyridinylcarbonyl)-D-lysyl-L-leucyl-N6(I-methylethyl)-L-lysyl-L-prolyl. Another example of a useful LHRH antagonist is GANIRELIX© (Roche/Akzo Nobel), hose chemical name is N-Ac-D-Nal,D-pCl-Phe,D-Pal,D-hArg(Et)₂,hArg(Et)₂,D-Ala. Examples of steroidal anti-androgens are cyproterone acetate (CPA) and megestrol acetate, commercially available as MEGACE© (Bristol-Myers Oncology). Steroidal anti-androgens may block prostatic androgen receptors. They may also inhibit the release of LH. CPA is preferably administered to human patients at dosages of 100 mg/day to 250 mg/day. Nonsteroidal anti-androgens block androgen receptors. They may also cause an increase in serum LH levels and Serum testosterone levels. A preferred nonsteroidal anti-androgen is flutamide (2-methyl-N-[4-20 nitro-3-(trifluoromethyl)phenyll propanamide), commercially available as EULEXIN© (Schering Corp.). Flutamide exerts is anti-androgenic action by inhibiting androgen uptake, by inhibiting nuclear binding of androgen in target tissues, or both. Another non-steroidal anti-androgen is nilutamide, whose chemical name is 5,5-dimethyl-3-[4-nitro-3-(trifluoromethyl-4′-nitrophenyl)-4,4-dimethyl-imidazolidine-dione.

In some embodiments of the invention, the anti-hormonal agent is a combination of an LHRH agonist such as leuprolide acetate, and an antiandrogen such as flutamide or nilutamide. For example, leuprolide acetate can be administered by subcucaneous, intramuscular or intravenous injection, and concurrently the flutamide can be administered orally.

Anti-hormonal agents according to the invention include, as pointed out above, antagonists of the steroid/thyroid hormone receptors, including antagonists for other non-permissive receptors, such as antagonists for RAR, TR, VDR, and the like. As readily recognized by those of skill in the art, a variety of retinoic acid receptor (RAR) antagonists, both synthetic and naturally occurring, can be used in accordance with the present invention. Examplary RAR antagonists include dicarba-closo-dodecaboranes (lijima et al., Chem Pharm Bull (Tokyo) (1999) 47(3):398-404), hydroanthracenyl, benzochromenyl and benzothiochromenyl retinoids (Vuligonda et al., Bioorg Med Chem Lett (1999) 9(5):743-8), diarylacetylenes, benzoic acid derivatives (see, e.g., Kagechika, H. (1994) Yakugaku Zasshi 114(11):847-862; Eckhardt et al. (1994) Toxicol Lett 70(3):299-308; Yoshimura et al. (1995) J Med Chem 38(16):3163-3173; 30 Chen et al. (1995) EMBO 14(6):1187-1197; Teng et al. (1997) J Med Chem 40(16):2445-2451); naphthalenyl analogs (see, e.g., Johnson et al. (1995) J Med Chem 38(24):4764-4767; Agarwal et al. J Biol Chem 271(21): 12209-12212: Umemiya et al. (1996) Yakugaku Zasshi 116(12):928-941); aryl-substituted and aryl and (3-oxo-l-propenly)-substituted benzopyran, benzothiopyran, 1,2-dihydroquinoline, and 5,6-dihydronaphthalene derivatives (Klein et al. U.S. Pat. Nos. 5877,207 and 5,776,699), adamantyl-substituted biaromatic compounds (Bernardon and Charpentier, U.S. Pat. No. 5,877,342), 1-phenyl-adamancane derivatives (Bernardon and Bernardon EP 776885), polyaromatic heterocyclic compounds (Charpentier et al. U.S. Pat. No. 5,849,798), dihydronaphthalene derivatives (Beard et al., U.S. Pat. No. 5,808,124 and Johnson et al. U.S. Pat. No. 5,773,594), 4-phenyl (benozopyranoyl or naphthoyl) amidobenzoic acid derivatives (WO 98/US/13065), diazepinylbenzoic acid derivatives (Umemiya et al., J Med Chem (1997) 40(26):4222-34), tetrahydronaphthalene derivatives (U.S. Pat. No. 5,763,635, 5,741,896 and 5,723,666), aryl-and heteroarylcyclohexenyl substituted alkenes (U.S. Pat. No. 5,760,276), dibenzofuran compounds, including aromatic dibenzofuran compounds (U.S. Pat. No. 5,702,710, U.S. Pat. No. 5,747,530), N-aryl substituted tetrahydroquinolines (U.S. Pat. No. 5,739,338), benzo[1,2-g]-Chrom-3-ene and benzo[1,2-g)-thiochrom-3-ene derivatives (U.S. Pat. No. 5,728,846), and the like. Examples of specific RAR antagonists contemplated for use herein include LE135 (Umemiya et al. (1996) Yakugaku Zasshi 116(12):928-941), LE511, LE540, LE550 (Li et al., J Biol Chem (1999) 274(22): 15360-6; Umemiya et al. (1996) Yakugaku Zasshi 116(12):928-941), Ro41-5253 (Keidel et al. (1994) Mol Gell Biol 14(1):287-298), SR11330, SR11334, SR11335 (Lee et al. (1996) J. Biol Chem 271(20):11897-11903), BMS453, BMS411 (Chen et al. (1995) EMBO 14(6):1187-1197), CD2366 and CD2665 (Meister et al., Anticancer Res. (1998) 18(3A): 1777-1786), ER27191 (Uemo et al., Leuk. Res. (1998) 22(6):517-525), AGN 193 109 (Johnson et al., Bioorg Med Chem Lett (1999) 9(4):5736), 4-[4,5,7,8,9,10-hexahydro-7,7,10,10-tetramethyl-1-(3-pyridylmethyl)anthra [1,2-b]pyrrol-3-yl]benzoic acid, 4-[4,5,7,8,9,10-hexahydro-7,7,10,10-tetramethyl-1-(3-pyridylmethyl)-5-thiaanthra[1,2-b]pyrrol-3-yl]benzoic acid, 4-[4,5,7,8,9,10-hexahydro-7,7,10,10-tetramethyl-1-(3-pyridylmethyl)anthra[2,1-d]pyrazol-3-yl]benzoic acid (Yoshimura et al. (1995) J Med Chem 38(16):3163-3173), AGN193109 (Agarwal et al. J Biol Chem 271 (21):12209-12212), and the like.

By “steroid receptor” or “nuclear steroid receptor ” is meant a protein that is a ligand-activated transcription factor, and belongs to the steroid receptor subfamily of nuclear receptors. Included in the definition of steroid receptors are proteins which structurally resemble and have the biological activity of a steroid hormone-activated transcription factor. Steroid receptors contain all or part of a DNA binding domain and a hormone (or ligand) binding domain, and include orphan receptors for unknown ligands whose structure resembles that of steroid receptors.

By “steroid receptor ligand” is meant a natural or synthetic compound which binds the nuclear steroid receptor to form a receptor-ligand complex. The term ligand includes agonists, antagonists, and compounds with partial agonist/antagonist action.

By “steroid receptor agonist” is meant a compound which binds the nuclear steroid receptor to form a receptor-agonist complex. The receptor-agonist complex binds specific regions of DNA termed hormone response elements. Agonists include steroid or steroid-like hormone, retinoids, thyroid hormones, pharmaceutically active compounds, and the like. Individual agonists may have the ability to bind to multiple receptors. Natural steroid hormone agonists include estradiol, progesterone, androgens, glucocorticoids, and mineralocorticoids. As pointed out above some steroid receptor agonists may show some efficiacy as antagonists as a function of dosage. Therefore, such an “agonist” may be effective as an anti-hormonal agent as defined according to this invention.

BY “steroid receptor antagonist” is meant a compound that has a biological effect opposite to that of an agonist. An antagonist binds the nuclear steroid receptor and blocks the action of a steroid receptor agonist by competing with the steroid agonist for receptor. An antagonist” is defined by its ability to block the actions of an agonist. Steroid receptor antagonists include “pure” antagonists, as well as compounds with partial agonist/antagonist action. A pure antagonist effectively competes with an agonist for receptor binding, without itself having agonist actions. A partial antagonist may be less effective at competing with an agonist for receptor binding, or may be equally effective at binding the receptor but have only 5-10% of the agonist action than that of the agonist being competed with. Thus, an antagonist may have an agonist effect less effective than that of the competing agonist.

“Radiotherapy”: According to the invention the tumors can additionally be treated with radiation or radiopharmaceuticals The source of radiation can be either external or internal to the patient being treated. When the source is external to the patient, the therapy is known as external beam radiation therapy (EBRT). When the source of radiation is internal to the patient, the treatment is called brachytherapy (BT). Some typical radioactive atoms that have been used include radium, cesium-137, and iridium-192, americium-241 and gold-198, Cobalt-57; Copper-67; Technetium-99; lodide-123; lodide-131; and Indium-111. It is also possible to label the agents according to the invention with radioactive isotopes.

Today radiation therapy is the standard treatment to control unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (rad), time and fractionation, and must be carefully defined by the oncologist. The amount of radiation a patient receives will depend on various consideration but the two most important considerations are the location of the tumor in relation to other critical structures or organs of the body, and the extent to which the tumor has spread. A preferred course of treatment for a patient undergoing radiation therapy will be a treatment schedule over a 5 to 6 week period, with a total dose of 50 to 60 Gy administered to the patient in a single daily fraction of 1.8 to 2.0 Gy, 5 days a week. A Gy is an abbreviation for Gray and refers to 100 rad of dose. In the preferred embodiment, there is synergy when tumors in human patients are treated with the angiogenesis antagonist and TNFα/IFNγ and radiation. In other words, the inhibition of tumor growth by means of said compounds is enhanced when combined with radiation and/or chemotherapeutic agents. Radiation therapy can be optionally used according to the invention. It is recommended and preferred in cases in which no sufficient amounts of the agents according to the invention can be administered to the patient.

“Pharmaceutical treatment”: The method of the invention comprises a variety of modalities for practicing the invention in terms of the steps. For example, the agents according to the invention can be administered simultaneously, sequentially, or separately. Furthermore, the agents can be separately administered within a time interval of about 3 weeks between administrations, i.e., from substantially immediately after the first active agent is administered to up to about 3 weeks after the first agent is administered. The method can be practiced following a surgical procedure. Alternatively, the surgical procedure can be practiced during the interval between administration of the first active agent and the second active agent. Exemplary of this method is the combination of the present method with surgical tumor removal. Treatment according to the method will typically comprise administration of the therapeutic compositions in one or more cycles of administration. For example, where a simultaneous administration is practiced, a therapeutic composition comprising both agents is administered over a time period of from about 2 days to about 3 weeks in a single cycle. Thereafter, the treatment cycle can be repeated as needed according to the judgment of the practicing physician. Similarly, where a sequential application is contemplated, the administration time for each individual therapeutic will be adjusted to typically cover the same time period. The interval between cycles can vary from about zero to 2 months.

The agents of this invention can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, the agents of this invention can be administered intraocularty, intravenously, intraperitoneally, intramuscularly, subcutaneously, intracavity, transdermally, by orthotopic injection and infusion, and can also be delivered by peristaltic means. The therapeutic compositions containing, for example, an integrin antagonist of this invention are conventionally administered intravenously, as by injection of a unit dose, for example. Therapeutic compositions of the present invention contain a physiologically tolerable carder together with the relevant agent as described herein, dissolved or dispersed therein as an active ingredient. As used herein, the term “pharmaceutically acceptable” refers to compositions, carriers, diluents and reagents which represent materials that are capable of administration to or upon a mammal without the production of undesirable physiological effects such as nausea, dizziness, gastric upset and the like. The preparation of a pharmacological composition that contains active ingredients dissolved or dispersed therein is well understood in the art and need not be limited based on formulation. Typically, such compositions are prepared as injectables either as liquid solutions or suspensions, however, solid forms suitable for solution, or suspensions, in liquid prior to use can also be prepared. The preparation can also be emulsified. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Particularly preferred is the HCl salt when used in the preparation of cyclic polypeptide αv antagonists. Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin vegetable oils such as cottonseed oil, and water-oil emulsions.

Typically, a therapeutically effective amount of an immunotherapeutic agent, for example, in the form of an integrin receptor blocking antibody or antibody fragment or antibody conjugate or an anti-VEGF receptor blocking antibody, fragment or conjugate is an amount such that when administered in physiologically tolerable composition is sufficient to achieve a plasma concentration of from about 0.01 microgram (μg) per milliliter (ml) to about 100 μg/ml, preferably from about 1 μg/ml to about 5 μg/ml and usually about 5 μg/ml. Stated differently the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily for one or several days. Where the immunotherapeutic agent is in the form of a fragment of a monoclonal antibody or a conjugate, the amount can readily be adjusted based on the mass of the fragment/conjugate relative to the mass of the whole antibody. A preferred plasma concentration in molarity is from about 2 micromolar (μM) to about 5 millimolar (mM) and preferably, about 100 μM to 1 mM antibody antagonist.

A therapeutically effective amount of an agent according of this invention which is a non-immunotherapeutic peptide or a protein polypeptide or other similarly-sized biological molecule, is typically an amount of polypeptide such that when administered in a physiologically tolerable composition is sufficient to achieve a plasma concentration of from about 0.1 microgram (bg) per milliliter (ml) to about 200 μg/ml, preferably from about 1 μg/ml to about 150 μg/ml. Based on a polypeptide having a mass of about 500 grams per mole, the preferred plasma concentration in molarity is from about 2 micromolar (μM) to about 5 millimolar (mM) and preferably about 100 μM to 1 mM polypeptide antagonist.

The typical dosage of an active agent, which is a preferably a nuclear hormone receptor antagonist or a (chemical) chemotherapeutic agent according to the invention (neither an immunotherapeutic agent nor a non-immunotherapeutic peptide/protein) is 10 mg to 1000 mg, preferably about 20 to 200 mg, and more preferably 50 to 100 mg per kilogram body weight per day.

The pharmaceutical compositions of this invention are preferably suitable for the treatment of breast and prostate cancer.

For breast cancer the following anti-hormonal agents and dosages are preferred:

| | |
|---|---|
| Tamoxifen: | 10.0 mg-40.0 mg p.o./day |
| Toremifen: | 20.0 mg-100.0 mg/day |
| Anastrozole: | 0.5 mg-5.0 mg p.o./day |
| Letrozole: | 1.0 mg-10.0 mg p.o./day |
| Formestane: | 100.0 mg-500.0 mg i.m. q 2 weeks |
| Goserelin: | 2.5 mg-5.0 mg s.c./4 weeks |
| Buserelin: | 5.0 mg-10.0 mg s.c. q 8 weeks |
| | 1.0 mg-5.0 mg nasal spray/d |
| Leuprorelin: | 5.0 mg-15.0 mg s.c. q 3 months |
| Megestrolacetate: | 40.0 mg-200.0 mg/d |

For breast cancer the following anti-hormonal agents and dosages are preferred:

| | |
|---|---|
| Flutamide: | 250.0 mg-1000.0 mg/d |
| Bicalutamide: | 10.0 mg-200.0 mg/d |
| Goserelin: | 2.5 mg-5.0 mg s.c./4 weeks |
| Buserelin: | 5.0 mg-10.0 mg s.c. q 8 weeks |
| | 1.0 mg-5.0 mg nasal spray 7 d |
| Leuprorelin: | 5.0 mg-15.0 mg s.c. q 3 months |
| Cyproteronacetate: | 25.0 mg-200.0 mg/d p.o. |
| | 150.0 mg-500.0 mg i.m. q 2 weeks |

Any analogs or further developments of the above-mentioned anti-hormone treatment principals will be used according to the proscribed doses.

The pharmaceutical compositions of the invention can comprise phrase encompasses treatment of a subject with agents that reduce or avoid side effects associated with the combination therapy of the present invention (“adjunctive therapy”), including, but not limited to, those agents, for example, that reduce the toxic effect of anticancer drugs, e.g., bone resorption inhibitors, cardioprotective agents. Said adjunctive agents prevent or reduce the incidence of nausea and vomiting associated with chemotherapy, radiotherapy or operation, or reduce the incidence of infection associated with the administration of myelosuppressive anticancer drugs. Adjunctive agents are well known in the art.

The immunotherapeutic agents according to the invention can additionally administered with adjuvants like BCG and immune system stimulators. Furthermore, the compositions may include immunotherapeutic agents or chemotherapeutic agents which contain cytotoxic effective radio labeled isotopes, or other cytotoxic agents, such as a cytotoxic peptides (e.g. cytokines) or cytotoxic drugs and the like.

The term “pharmaceutical kit” for treating tumors or tumor metastases refers to a package and, as a rule, instructions for using the reagents in methods to treat tumors and tumor metastases. A reagent in a kit of this invention is typically formulated as a therapeutic composition as described herein, and therefore can be in any of a variety of forms suitable for distribution in a kit. Such forms can include a liquid, powder, tablet, suspension and the like formulation for providing the antagonist and/or the fusion protein of the present invention. The reagents may be provided in separate containers suitable for administration separately according to the present methods, or alternatively may be provided combined in a composition in a single container in the package. The package may contain an amount sufficient for one or more dosages of reagents according to the treatment methods described herein. A kit of this invention also contains “instruction for use” of the materials contained in the package.

1. A pharmaceutical composition comprising in therapeutically effective amount at least (i) one anti-EGFR antibody or an immunotherapeutically effective fragment thereof and (ii) one anti-hormonal agent, optionally together with a pharmaceutically acceptable carrier, excipient, or diluent.

2-20. (canceled)
